(19) United States
(12) Patent Application Publication
(10) Pub. No.: US 2025/0025180 A1
(43) Pub. Date: Jan. 23, 2025

(54) EXPANDING FOAM DELIVERY SYSTEM FOR OCCLUDING LEFT ATRIAL APPENDAGE

(71) Applicant: BOSTON SCIENTIFIC SCIMED, INC., MAPLE GROVE, MN (US)

(72) Inventor: Patrick Willoughby, Shoreview, MN (US)

(73) Assignee: BOSTON SCIENTIFIC SCIMED, INC., MAPLE GROVE, MN (US)

(21) Appl. No.: 18/776,952

(22) Filed: Jul. 18, 2024

Related U.S. Application Data
(60) Provisional application No. 63/528,112, filed on Jul. 21, 2023.

(51) Int. Cl. A61B 17/12; A61B 17/00; A61B 18/00; A61B 18/14; A61M 25/09

(52) U.S. Cl. CPC A61B 17/12122; A61B 17/1219; A61B 18/1492; A61B 2017/00247; A61B 2017/00632; A61B 2017/00871; A61B 2017/1205; A61B 2018/00601; A61M 25/09

(57) ABSTRACT

A left atrial appendage closure device adapted for occluding the left atrial appendage includes a cone-shaped insert formed of an expandable foam and adapted for trans-septal delivery to the LAA. A polymeric covering is disposed over the cone-shaped insert, the polymeric covering adapted to protect the cone-shaped insert during trans-septal delivery to the LAA, the polymeric covering adapted for removal after trans-septal delivery to the LAA. An insert lumen extends through the cone-shaped insert prior to expansion of the cone-shaped insert. A tubular support rod extends through the insert lumen, the tubular support rod operably coupled with at least a portion of the polymeric covering, the tubular support rod itself defining a tubular support rod lumen adapted to accommodate a guidewire over which the insert may be advanced into the LAA and/or a radio frequency (RF) energy wire adapted for making a trans-septal puncture.

### CROSS REFERENCE TO RELATED APPLICATIONS

This application claims the benefit of priority of U.S. Provisional Application No. 63/528,112 filed Jul. 21, 2023, the entire disclosure of which is hereby incorporated by reference.

### TECHNICAL FIELD

The disclosure relates generally to medical devices and more particularly to medical devices that are adapted for detecting leaks around a left atrial appendage closure device.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device adapted for occluding the left atrial appendage (LAA). The LAAC device includes an implant adapted for trans-septal delivery to the LAA, the implant including an expandable foam. A polymeric covering is disposed over the implant and is adapted to protect the implant during trans-septal delivery to the LAA, the polymeric covering adapted for removal after trans-septal delivery to the LAA.

Alternatively or additionally, the LAAC device may further include an implant lumen extending through the implant prior to expansion of the implant and a tubular support rod extending through the implant lumen, the tubular support rod operably coupled with at least a portion of the polymeric covering. The tubular support rod defines a tubular support rod lumen adapted to accommodate one or more of a guidewire over which the implant may be advanced into the LAA and a radio frequency (RF) energy wire adapted for making a trans-septal puncture.

Alternatively or additionally, the implant may have a shape that facilitates trans-septal delivery to the LAA.

Alternatively or additionally, the implant may include an expandable foam that expands upon reaching a temperature elevated from ambient temperature.

Alternatively or additionally, the implant may include an expandable foam that expands upon exposure to moisture.

Alternatively or additionally, the proximal covering may include a distal region with a plurality of perforations formed within the polymeric covering, the plurality of perforations adapted to allow removal of the polymeric covering relative to the implant.

Alternatively or additionally, the polymeric covering and the tubular support rod may both be adapted to independently be pulled proximally in order to expose the implant.

Alternatively or additionally, the LAAC device may further include a break point formed in the polymeric covering, and pulling the tubular support rod proximally may cause the polymeric covering to sever at the break point, with a portion of the polymeric covering distal of the break point retracting with the tubular support rod through the implant lumen.

Alternatively or additionally, the proximal covering may be adapted to dissolve after trans-septal delivery to the LAA.

Alternatively or additionally, the LAAC device may further include one or more anchor features embedded within the implant for anchoring the LAAC device within the LAA.

Alternatively or additionally, the LAAC device may further include one or more tethers temporarily securing the implant to the tubular support rod.

Alternatively or additionally, the expandable foam may include a shape memory foam.

Another example may be found in a left atrial appendage closure (LAAC) device adapted for occluding the left atrial appendage (LAA). The LAAC device includes an expandable foam cone-shaped implant adapted for trans-septal delivery to the LAA and a cone-shaped covering disposed over the expandable foam cone-shaped implant, the cone-shaped covering adapted to protect the expandable foam cone-shaped implant during trans-septal delivery to the LAA, the cone-shaped covering including a distal region having a plurality of perforations adapted to split apart to facilitate removal of the cone-shaped covering. An insert lumen extends through the cone-shaped implant when in its delivery configuration. A tubular support rod extends through the implant lumen, the tubular support rod operably coupled with at least a portion of the cone-shaped covering, the tubular support rod itself defining a tubular support rod lumen extending therethrough.

Alternatively or additionally, the implant lumen may be adapted to disappear as the expandable foam cone-shaped implant expands.

Alternatively or additionally, the LAAC device may further include one or more anchor features embedded within the expandable foam cone-shaped implant for anchoring the LAAC device within the LAA.

Alternatively or additionally, the LAAC device may further include one or more tethers temporarily securing the expandable foam cone-shaped implant to the tubular support rod.

Another example may be found in a left atrial appendage closure (LAAC) device adapted for occluding the left atrial appendage (LAA). The LAAC device includes a shape memory foam implant adapted for trans-septal delivery to the LAA and a cone-shaped covering disposed over the shape memory foam insert, the cone-shaped covering adapted to protect the shape memory foam implant during trans-septal delivery to the LAA, the cone-shaped covering including a distal region having a plurality of perforations adapted to split apart to facilitate removal of the cone-shaped covering. An implant lumen extends through the shape memory foam implant when in its delivery configuration. A tubular support rod extends through the implant lumen and is operably coupled with at least a portion of the cone-shaped covering, the tubular support rod itself defining a tubular support rod lumen extending therethrough. The shape memory foam implant is adapted to expand in diameter upon implantation within the LAA.

Alternatively or additionally, the LAAC device may further include one or more anchor features embedded within the shape memory foam implant for anchoring the LAAC device within the LAA.

Alternatively or additionally, the LAAC device may further include one or more tethers temporarily securing the shape memory foam implant to the tubular support rod.

Alternatively or additionally, the shape memory foam implant may be adapted to expand into the LAA.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a schematic view of an LAAC (left atrial appendage closure) device;

FIG. 3 is a cross-sectional view taken along the line 3-3 of FIG. 2;

FIG. 4 is a schematic view of the LAAC device of FIG. 2, in a partially expanded state;

FIG. 5 is a cross-sectional view taken along the line 5-5 of FIG. 4;

FIG. 6 is a schematic view of the LAAC device of FIG. 2, at a further expanded state;

FIG. 7 is a cross-sectional view taken along the line 7-7 of FIG. 6;

FIGS. 8 through 11 are schematic views of the LAAC device of FIG. 2 deployed within the LAA of FIG. 1, at increasing degrees of expansion;

FIG. 12 is a schematic view of an LAAC (left atrial appendage closure) device;

FIG. 13 is a cross-sectional view taken along the line 13-13 of FIG. 12; and

FIG. 14 is a schematic view of an illustrative delivery device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

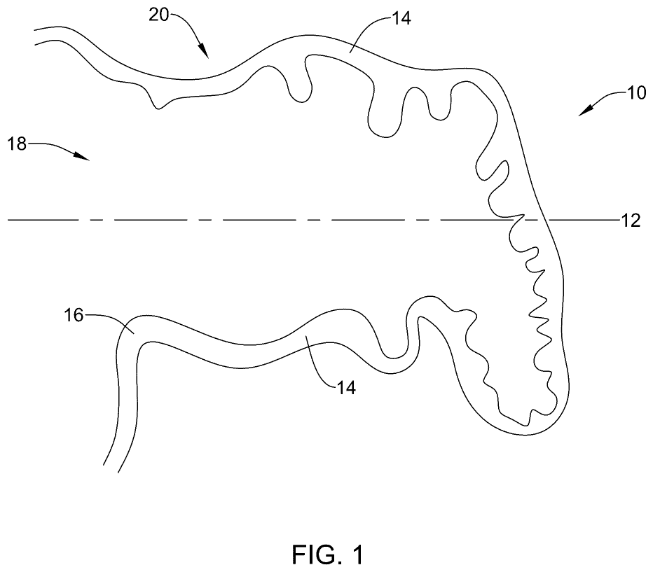

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like clement associated with a distal portion of the main body 20. In some examples, the distalmost region may protrude radially or laterally away from the longitudinal axis 12.

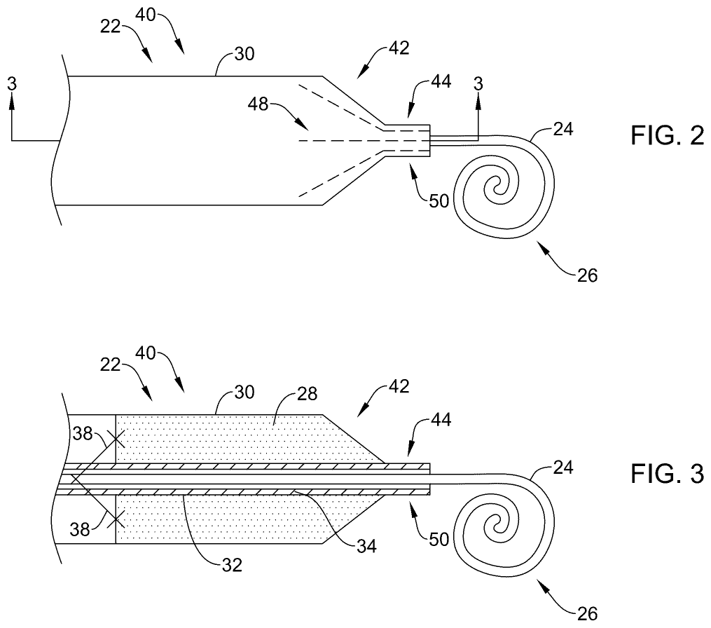

In some instances, the LAA 10 may be reached via a trans-septal approach in which LAA 10 is reached from the right atrium, passing through the atrial septum, and into the left atrium. It will be appreciated that this may be considered as being an endocardial approach, which may be considered less invasive than an epicardial approach. In some instances, a trans-septal approach may include reaching the right atrium, and then using a guidewire or an RF (radio frequency) energy wire to puncture the atrial septum. From there, the LAA 10 may be reached by passing through the atrial septum and into the left atrium. FIG. 2 is a schematic view of an illustrative LAAC (left atrial appendage closure) device 22 that may be delivered via a trans-septal approach and used in closing off the LAA 10. FIG. 3 is a cross-sectional view of the LAAC device 22, taken along the line 3-3 of FIG. 2. In some instances, the LAAC device 22 may be advanced through the right atrium, through the atrial septum, through the left atrium and into the LAA 10 over a guidewire 24. In some instances, the guidewire 24 may be adapted to provide an atraumatic distal tip by forming a pigtail 26. In some instances, the guidewire 24 may not form the pigtail 26, and thus may not be considered as including an atraumatic distal tip.

The LAAC device 22 includes an implant 28 that is adapted for trans-septal delivery to the LAA 10. In some instances, the implant 28 may have a shape or overall profile that facilitates a trans-septal delivery to the LAA 10. In some instances, the implant 28 may have a tapered profile having a minimal diameter at a distal end of the implant 28 that expands to a larger diameter closer to a proximal end of the implant 28. An example of a tapered profile would be a cone-shape. It will be appreciated that reference to the profile of the implant 28 generally refers to the collapsed configuration of the implant 28, as the implant 28 will likely lose this distinctive shape upon expansion.

In some instances, the implant 28 may be formed of or otherwise include an expandable foam. In some instances, the expandable foam may be or otherwise include a shape memory foam that may be adapted to revert to a remembered shape upon application of, or exposure to, one or more appropriate stimulations. As an example, a shape memory foam may expand to a remembered expanded configuration upon exposure to moisture (such as blood) or temperatures (such as body temperatures) that are elevated relative to ambient temperature outside of the patient. In some instances, a shape memory foam may expand upon exposure to a combination of moisture and elevated temperature. In some instances, a shape memory foam may expand to a diameter that is up to, or even more than, ten times its diameter before expansion, which may be referred to as a 1:10 expansion. In some instances, the shape memory foam may expand considerably more than this.

In some instances, the implant 28 may itself be sufficiently rigid to withstand a trans-septal crossing. In some instances, the LAAC device 22 may include a polymeric covering 30 that spans the implant 28. The polymeric covering 30 may be adapted to provide additional rigidity to the LAAC device 22. In some instances, the polymeric covering 30 may be adapted to help protect the implant 28 from exposure to blood during advancement of the LAAC device 22 to the LAA 10. The polymeric covering 30 may be formed of any suitable polymeric materials. Illustrative examples include semi-crystalline or crystalline polymers like polyethylene terapthalate (PET), polypropylene (PP), ultrahigh molecular weight polyethylene (UHMWPE), polytetrafluoroethylene (PTFE), polyetheretherketone (PEEK), polyamides (nylon), or thermoplastic polyesters like polylactic acid (PLA, PLLA, or PLGA); amorphous polymers like polycarbonate (PC), polymethylmethacrylate (PMMA); or complex thermoplastic elastomers like polyurethanes (PUR—various suitable compositions) or polyether block amides (PEBAX).

In a non-expanded configuration, as shown for example in FIG. 3, the implant 28 defines an insert lumen 32 that extends axially through the cone-shape implant 28. It will be appreciated that in some instances, once the implant 28 begins to expand, that expansion of the foam forming the implant 28 may obscure or fill in the implant lumen 32. A tubular support rod 34 is adapted to extend within the implant lumen 32, and itself defines a tubular support rod lumen 36 that extends through the tubular support rod 34. The tubular support rod 34 may be formed of any suitable metallic or polymeric material. As an example, the tubular support rod 34 may include or be formed from a hypotube or a more compliant polymeric tubing. The tubular support rod 34, when positioned within the implant lumen 32, may be considered as being adapted to accommodate the guidewire 24 extending through the tubular support rod lumen 36. When the guidewire 24 is not positioned within the tubular support rod lumen 36, it will be appreciated that an RF energy wire (not shown) may be extended through the tubular support rod lumen 36 in order to puncture the atrial septum.

In some instances, the LAAC device 22 may include one or more tethers 38 that extend from the implant 28 to the tubular support rod 34. The one or more tethers 38 may be formed of any suitable material, such as surgical thread, and may be adapted to help hold the implant 28 in position relative to the tubular support rod 34 until such as time as the tubular support rod 34 is withdrawn proximally. In some instances, as will be discussed, the LAAC device 22 may include one or more anchor or fixation members (not seen in FIG. 2 or 3) that can assist in holding the LAAC device 22 in position before the implant 28 fully expands.

In some instances, the LAAC device 22 defines a constant diameter region 40 in which the polymeric covering 30 (and the implant 28 therein) has a constant diameter, a tapered region 42 and a distal tip region 44. The tapered region 42 tapers distally from the constant diameter region 40 to the distal tip region 44. In some instances, the tubular support rod 34 may be secured to the polymeric covering 30 within the distal tip region 44. It will be appreciated that the distal tip region 44 has a minimal diameter that accommodates the tubular support rod 34 while still presenting a minimal face (along with the tapered region 42) for advancing the LAAC device 22 through the vasculature in reaching the LAA 10.

In some instances, as shown for example in FIG. 2, the polymeric covering 30 may include a pattern of perforations 46 that are adapted to allow the polymeric covering 30 to open up and be withdrawn proximally in order to expose the implant 28 to the body temperature blood within the LAA 10. In some instances, the pattern of perforations 46 include a first set of perforations 48 that are disposed within the tapered region 42 and a second set of perforations 50 that are disposed within the distal tip region 44. In some instances, the perforations 46 may extend all the way through a thickness of the polymeric covering 30. In some instances, the perforations 46 may only extend part way through the thickness of the polymeric covering 30, and may represent weak points at which the polymeric covering 30 will preferentially break upon application of sufficient force. Alternatively, in some instances, the polymeric covering 30 may be adapted to dissolve after a certain period of time exposed to blood, for example, or a solution to dissolve the polymeric covering 30 may be injected into the LAA 10.

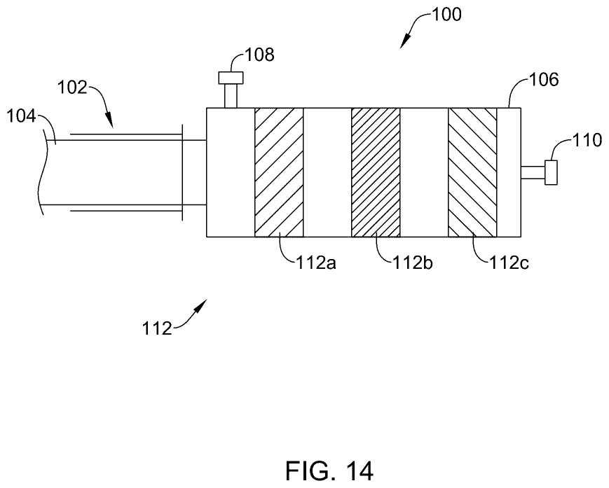

It will be appreciated that only the distal portions of the LAAC device 22 are shown. In some instances, the LAAC device 22 may include a proximal handle that would include features allowing for controlling deployment of the cone-shaped implant 28. A proximal handle may include features for allowing disconnection of the cone-shaped implant 28. In some instances, a proximal handle may allow for engagement of any anchoring features. A delivery system may include such a proximal handle, and may also include features to allow for system flushing and injection of contrast media, for example. A delivery system may be steerable, for example. An example delivery system is referenced in FIG. 14.

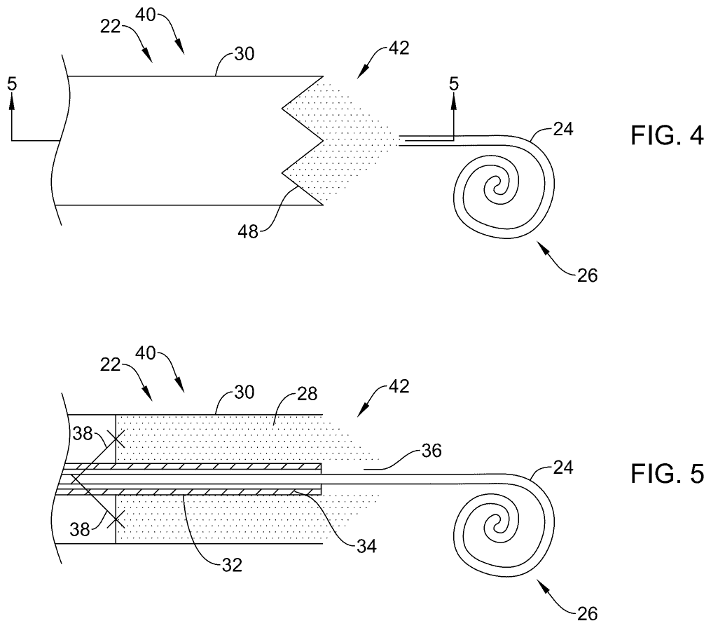

In some instances, the proximal covering 30 and the tubular support rod 34 may both be withdrawn proximally in order to begin uncovering the implant 28, thereby exposing the foam forming the implant 28 to the body temperature blood within the LAA 10. In FIG. 4, which is a schematic view of the LAAC device 22, and FIG. 5, which is a cross-sectional view taken along the line 5-5 of FIG. 4, it can be seen that the polymeric covering 30 has been severed at the first set of perforations 48. The second set of perforations 50 have also been severed, thereby releasing the tubular support rod 34 from the distal tip region 44. In some instances, applying a tensile force to the polymeric covering 30 may cause the perforations 48 to sever. As can be seen, the cone-shaped implant 28 is beginning to be exposed to the environment outside of the polymeric covering 30 as the tapered region 42 is now distal of the remaining polymeric covering 30.

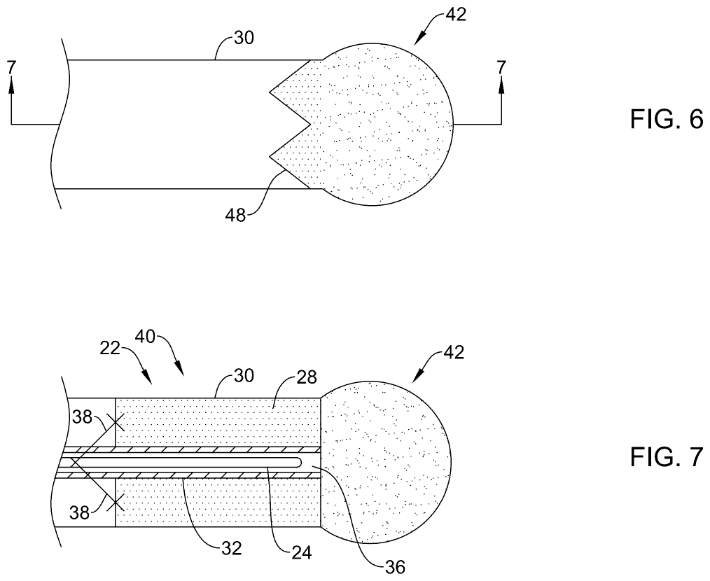

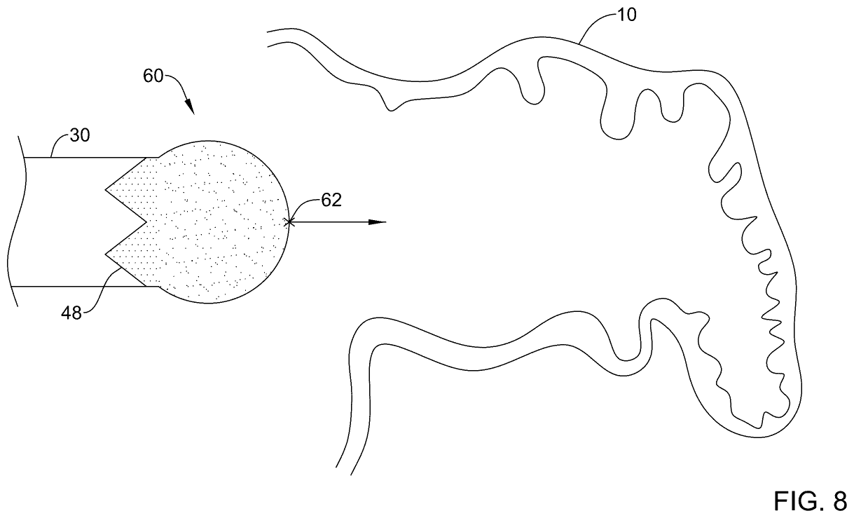

FIG. 6 is a schematic view of the LAAC device 22 and FIG. 7 is a cross-sectional view of the same, taken along the line 7-7 of FIG. 6. In FIG. 6, it can be seen that the previously tapered region 42 has begun to expand in diameter. The tubular support rod 34 has been pulled back somewhat but remains within the insert lumen 32, as does the guidewire 24. The one or more tethers 38 still secure the implant 28 relative to the tubular support rod 34. FIG. 8 is similar to FIG. 6, but shows the LAAC device 22 disposed proximate the LAA 10. As seen, the LAAC device 22 includes an expanded portion 60 that has resulted from part of the implant 28 expanding as a result of exposure to body temperature blood in and near the LAA 10. In some instances, as shown, the LAAC device 22 includes an anchor 62 extending distally from the expanded portion 60. While a single anchor 62 is shown, in some instances there may be two, three or more anchors 62. In some instances, the anchor 62 may be embedded within the cone-shaped implant 28, and expansion of the implant 28 may cause the anchor 62 to extend outwardly from the expanded portion 60.

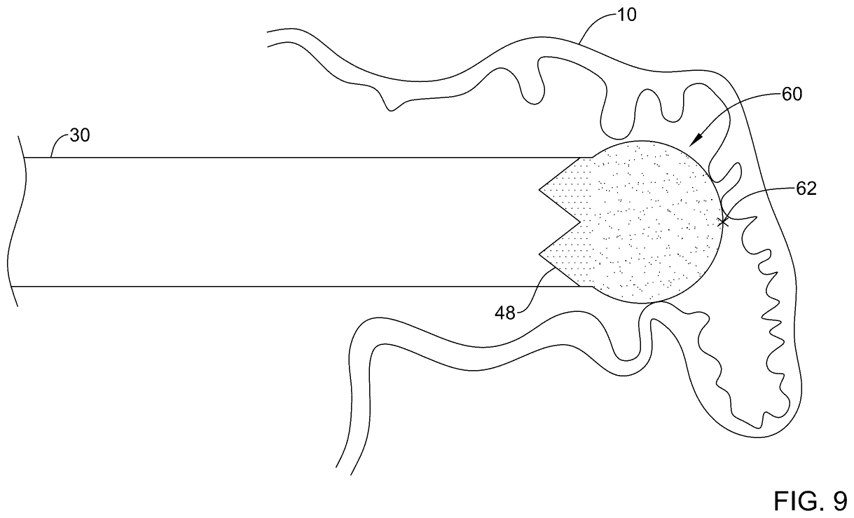

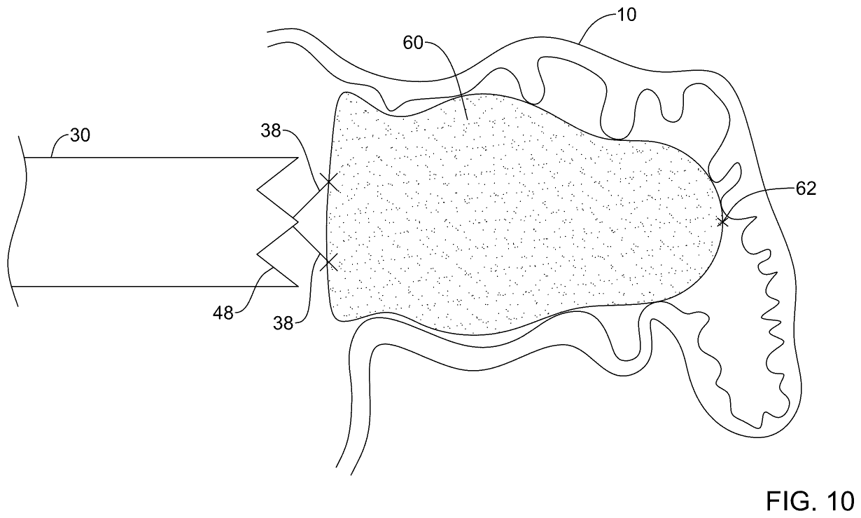

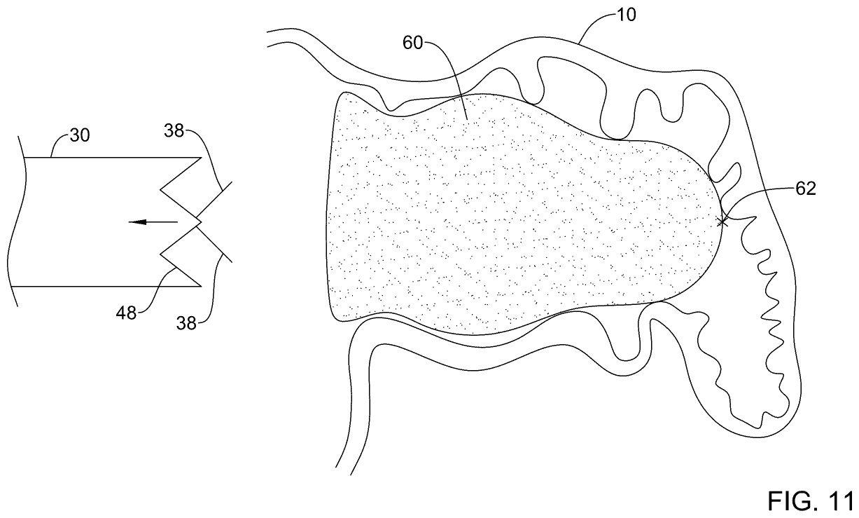

In FIG. 9, the LAAC device 22 has been advanced distally into the LAA 10 such that the anchor 62 has engaged the wall of the LAA 10. In some instances, the anchor 62 will help to hold the LAAC device 22 in position relative to the LAA 10 until the implant 28 has finished expanding. Moving to FIG. 10, it can be seen that the expanded portion 60 has further expanded, and is now consuming a greater fraction of the volume within the LAA 10. The tethers 38 still secure the implant 28 in position relative to the tubular support rod 32 (not visible in this view). In FIG. 11, parts of the LAAC device 22 distinct from the implant 28 are being withdrawn proximally. This will sever the tethers 38 that were holding the implant 28 to the tubular support rod 32, which is being withdrawn proximally along with the polymeric covering 30.

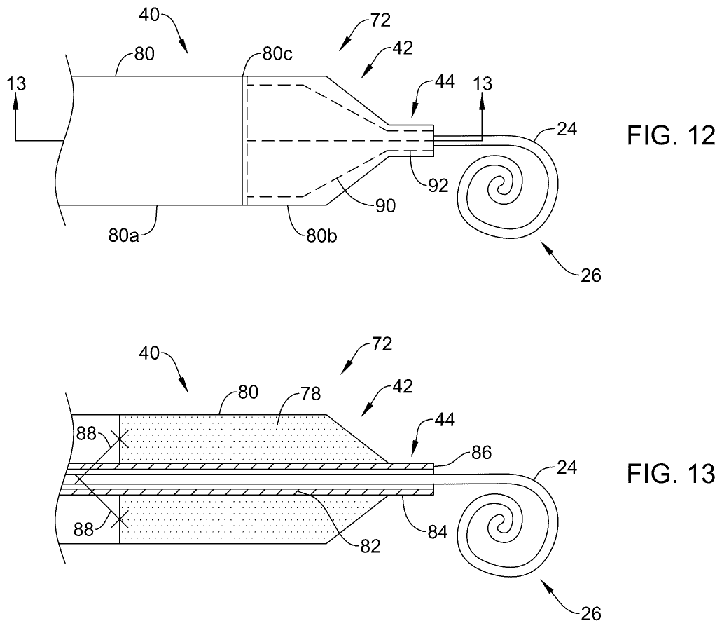

FIG. 12 is a schematic view of an illustrative LAAC (left atrial appendage closure) device 72 that may be delivered via a trans-septal approach and used in closing off the LAA 10. FIG. 13 is a cross-sectional view of the LAAC device 72, taken along the line 13-13 of FIG. 12. In some instances, the LAAC device 72 may be advanced through the right atrium, through the atrial septum, through the left atrium and into the LAA 10 over the guidewire 24. In some instances, the guidewire 24 may be adapted to provide an atraumatic distal tip by forming the pigtail 26. In some instances, the guidewire 24 may not form the pigtail 26, and thus may not be considered as including an atraumatic distal tip.

The LAAC device 72 includes an implant 78 that is adapted for trans-septal delivery to the LAA 10. In some instances, the implant 78 may be formed of or otherwise include an expandable foam. In some instances, the expandable foam may be or otherwise include a shape memory foam that may be adapted to revert to a remembered shape upon application of, or exposure to, one or more appropriate stimulations. As an example, a shape memory foam may expand to a remembered expanded configuration upon exposure to moisture (such as blood) or temperatures (such as body temperatures) that are elevated relative to ambient temperature outside of the patient. As another example, a shape memory foam may expand upon exposure to a combination of moisture and elevated temperature. In some instances, this exposure may provide a number of benefits, including a reduced size for delivery yet being able to expand considerably to occlude a large orifice such as the LAA 10. In some instances, a shape memory foam may exhibit a controlled initiation of expansion, i.e., the shape memory foam won't start to expand until exposed to moisture and or elevated temperature within the patient.

In some instances, the implant 78 may itself be sufficiently rigid to withstand a trans-septal crossing. In some instances, the LAAC device 72 may include a polymeric covering 80 that spans the implant 78. The polymeric covering 80 may be adapted to provide additional rigidity to the LAAC device 72. In some instances, the polymeric covering 80 may be adapted to help protect the implant 78 from exposure to blood during advancement of the LAAC device 22 to the LAA 10. The polymeric covering 80 may be formed of any suitable polymeric materials. Illustrative examples include those discussed with respect to the polymeric covering 30.

In a non-expanded configuration, as shown for example in FIG. 13, the implant 78 defines an insert lumen 82 that extends axially through the implant 78. It will be appreciated that in some instances, once the implant 78 begins to expand, that expansion of the foam forming the implant 78 may obscure or fill in the implant lumen 82. A tubular support rod 84 is adapted to extend within the implant lumen 82, and itself defines a tubular support rod lumen 86 that extends through the tubular support rod 84. The tubular support rod 84 may be formed of any suitable metallic or polymeric material. As an example, the tubular support rod 84 may include or be formed from a hypotube or polymeric tube. The tubular support rod 84, when positioned within the implant lumen 82, may be considered as being adapted to accommodate the guidewire 24 extending through the tubular support rod lumen 86. When the guidewire 24 is not positioned within the tubular support rod lumen 86, it will be appreciated that an RF energy wire (not shown) may be extended through the tubular support rod lumen 86 in order to puncture the atrial septum.

In some instances, the LAAC device 22 may include one or more tethers 88 that extend from the implant 78 to the tubular support rod 84. The one or more tethers 88 may be formed of any suitable material, such as surgical thread, and may be adapted to help hold the implant 78 in position relative to the tubular support rod 84 until such as time as the tubular support rod 84 is withdrawn proximally. In some instances, the LAAC device 72 may include one or more anchor or fixation members (not seen in FIG. 2 or 3) that can assist in holding the LAAC device 22 in position before the implant 78 fully expands.

In some instances, the LAAC device 22 defines a constant diameter region 40 in which the polymeric covering 80 (and the implant 78 therein) has a constant diameter, a tapered region 42 and a distal tip region 44. The tapered region 42 tapers distally from the constant diameter region 40 to the distal tip region 44. In some instances, the tubular support rod 84 may be secured to the polymeric covering 80 within the distal tip region 44. It will be appreciated that the distal tip region 44 has a minimal diameter that accommodates the tubular support rod 84 while still presenting a minimal face (along with the tapered region 42) for advancing the LAAC device 72 through the vasculature in reaching the LAA 10.

In some instances, the polymeric covering 80 may include a first region 80a and a second region 80b, with a break point 80c disposed between the first region 80a and the second region 80b. In some instances, the first region 80a may be adapted to be withdrawn proximally along an exterior of the cone-shaped implant 78 while the second region 80b may be adapted to pulled proximally within the implant lumen 82 along with the tubular support rod 84. The second region 80b includes a first set of perforations 90 while the distal tip region 44 includes a second set of perforations 92. In some instances, the perforations 90 and 92 may extend all the way through a thickness of the polymeric covering 30. In some instances, the perforations 90 and 92 may only extend part way through the thickness of the polymeric covering 80, and may represent weak points at which the polymeric covering 80 will preferentially break upon application of sufficient force.

FIG. 14 is a schematic view of an illustrative delivery system 100. The illustrative delivery system 100 includes an integrated introducer feature 102 that extends over an elongate shaft 104. The elongate shaft 104 extends distally from a handle 106. It will be appreciated that a distal region of the elongate shaft 104 may be adapted to accommodate and releasably engage the LAAC device 22. In some instances, the handle 106 may include an external flushing port 108 that allows a flushing fluid such as saline to be introduced outside of the central lumen 32, for example. In some instances, the handle 106 may include an internal flushing port 110 that allows a flushing fluid such as saline to be introduced inside of the central lumen 32.

The handle 106 also includes a number of control elements 112, individually labeled as 112a, 112b and 112c. While a total of three control elements 112 are shown, it will be appreciated that in some instances, the handle 106 may include fewer than three control elements 112. In some instances, the handle 96 may include four, five or more control elements 112. Each of the control elements 112 may each be a knob, a lever, a slide or a pull that affects a motion or action at the distal end. Examples of motions or actions at the distal end include but are not limited to retracting and extending an outer housing feature, retracting and extending the central lumen 32, or causing the central lumen 32 to move concurrently with the outer housing feature, retracting the outer housing inside the central lumen 32, actuating an anchoring mechanism, and actuating a disconnection mechanism between the delivery system 100 and the LAAC device 22. These are just examples.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, from about 37° C. to about 50° C., including from about 37° C. to about 40° C., which allows the shape memory foam to assume an initial configuration prior to and during delivery through a delivery catheter or other delivery device, and an expanded configuration for occlusion after delivery and release within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ to about 1000 kg/m³, including from about 10 kg/m³ to about 500 kg/m³, including from about 10 kg/m³ to about 200 kg/m³, including from about 20 kg/m³ to about 100 kg/m³.

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha, omega-bis(p-carboxyphenoxy)alkane anhydride] such as poly [1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others.

A shape memory foam may have a thermal transition point (transition temperature) below which residual stress is maintained without a loading constraint. The thermal activation (which causes the shape memory) may be achieved with the desired material going through a semi-crystalline melt point or glass transition temperature between the first configuration and the expanded configuration. Several suitable thermal activation processes are known in the art and useful herein. In an example, the temperature activated memory shape foam may be formed for use as a medical device by first shaping a shape memory foam formed from a suitable material into its final expanded configuration; that is, the configuration that the shape memory shape foam will achieve once inserted into the left atrial appendage to provide the desired occlusive benefit. In this expanded configuration, the shape memory foam may generally have a diameter that will range from about 10 millimeters to about 50 millimeters and a length that will range from about 1 centimeter to about 5 centimeters, although other diameters and lengths are within the scope of the present disclosure. Once this has been done, the foam may be heated above the transition temperature of the material; that is, the temperature at which a desired expansion will occur. As noted above, suitable transition temperatures may be from about 37° C. to about 50° C. Once the desired transition temperature has been achieved, the shape memory foam is held at a constant temperature and is re-shaped into an initial (unexpanded) configuration. This re-shaping is suitably done in a properly sized molding clement and may be any suitable shape. In this collapsed configuration, the shape memory foam will generally have a diameter of less than 4.7 millimeters and a length that will range from about 2 centimeters to about 5 centimeters, although other diameters and lengths are within the scope of the present disclosure. After insertion into the molding element, the temperature is reduced to a temperature below the transition temperature to set the new shape; for example, the temperature may be reduced to room temperature to set the new shape. Once this has been completed, the shape memory foam will remain in its first configuration until it is subjected to a temperature at or above the transition temperature, at which time it will expand into its expanded, or remembered, configuration. In some instances, exposure to water within the blood changes the glass transition temperature of the foam. As an example, the shape memory foam may have a dry T_{g} (glass transition temperature) that is above body temperature, and may have a wet T_{g}, after exposure to water, that is lower than body temperature.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding the left atrial appendage (LAA), the LAAC device comprising:
an implant adapted for trans-septal delivery to the LAA, the implant comprising an expandable foam; and
a polymeric covering disposed over the implant, the polymeric covering adapted to protect the implant during trans-septal delivery to the LAA, the polymeric covering adapted for removal after trans-septal delivery to the LAA.

2. The LAAC device of claim 1, further comprising:
an implant lumen extending through the implant prior to expansion of the implant; and
a tubular support rod extending through the implant lumen, the tubular support rod operably coupled with at least a portion of the polymeric covering, the tubular support rod itself defining a tubular support rod lumen adapted to accommodate one or more of:
a guidewire over which the implant may be advanced into the LAA;
a radio frequency (RF) energy wire adapted for making a trans-septal puncture.

3. The LAAC device of claim 1, wherein the implant has a shape that facilitates trans-septal delivery to the LAA.

4. The LAAC device of claim 1, wherein the implant comprises an expandable foam that expands upon reaching a temperature elevated from ambient temperature.

5. The LAAC device of claim 1, wherein the implant comprises an expandable foam that expands upon exposure to moisture.

6. The LAAC device of claim 1, wherein the proximal covering includes a distal region with a plurality of perforations formed within the polymeric covering, the plurality of perforations adapted to allow removal of the polymeric covering relative to the implant.

7. The LAAC device of claim 2, wherein the polymeric covering and the tubular support rod are both adapted to independently be pulled proximally in order to expose the implant.

8. The LAAC device of claim 2, further comprising a break point formed in the polymeric covering, and pulling the tubular support rod proximally causes the polymeric covering to sever at the break point, with a portion of the polymeric covering distal of the break point retracting with the tubular support rod through the implant lumen.

9. The LAAC device of claim 1, wherein the proximal covering is adapted to dissolve after trans-septal delivery to the LAA.

10. The LAAC device of claim 1, further comprising one or more anchor features embedded within the implant for anchoring the LAAC device within the LAA.

11. The LAAC device of claim 1, further comprising one or more tethers temporarily securing the implant to the tubular support rod.

12. The LAAC device of claim 1, wherein the expandable foam comprises a shape memory foam.

13. A left atrial appendage closure (LAAC) device adapted for occluding the left atrial appendage (LAA), the LAAC device comprising:
an expandable foam cone-shaped implant adapted for trans-septal delivery to the LAA;
a cone-shaped covering disposed over the expandable foam cone-shaped implant, the cone-shaped covering adapted to protect the expandable foam cone-shaped implant during trans-septal delivery to the LAA, the cone-shaped covering including a distal region having a plurality of perforations adapted to split apart to facilitate removal of the cone-shaped covering;
an insert lumen extending through the cone-shaped implant when in its delivery configuration; and
a tubular support rod extending through the implant lumen, the tubular support rod operably coupled with at least a portion of the cone-shaped covering, the tubular support rod itself defining a tubular support rod lumen extending therethrough.

14. The LAAC device of claim 13, wherein the implant lumen is adapted to disappear as the expandable foam cone-shaped implant expands.

15. The LAAC device of claim 13, further comprising one or more anchor features embedded within the expandable foam cone-shaped implant for anchoring the LAAC device within the LAA.

16. The LAAC device of claim 13, further comprising one or more tethers temporarily securing the expandable foam cone-shaped implant to the tubular support rod.

17. A left atrial appendage closure (LAAC) device adapted for occluding the left atrial appendage (LAA), the LAAC device comprising:
a shape memory foam implant adapted for trans-septal delivery to the LAA;
a cone-shaped covering disposed over the shape memory foam insert, the cone-shaped covering adapted to protect the shape memory foam implant during trans-septal delivery to the LAA, the cone-shaped covering including a distal region having a plurality of perforations adapted to split apart to facilitate removal of the cone-shaped covering;
an implant lumen extending through the shape memory foam implant when in its delivery configuration; and
a tubular support rod extending through the implant lumen, the tubular support rod operably coupled with at least a portion of the cone-shaped covering, the tubular support rod itself defining a tubular support rod lumen extending therethrough;
wherein the shape memory foam implant is adapted to expand in diameter upon implantation within the LAA.

18. The LAAC device of claim 17, further comprising one or more anchor features embedded within the shape memory foam implant for anchoring the LAAC device within the LAA.

19. The LAAC device of claim 17, further comprising one or more tethers temporarily securing the shape memory foam implant to the tubular support rod.

20. The LAAC device of claim 17, wherein the shape memory foam implant is adapted to expand into the LAA.
